SEQUENCE LISTING (46 sequences)

SEQ ID NO: 1 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 2 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 3 - protein, 162 aa, Bos taurus
LIVTQTMKGLDIQKVAGTWYSLAMAASDISLLDAQSAPLRVYVEELKPTPEGDLEILLHKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 4 - protein, 162 aa, Ovis aries
IIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 5 - protein, 162 aa, Capra hircus
IIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 6 - protein, 162 aa, Bubalus bubalis
IIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 7 - protein, 162 aa, Equus caballus
TNIPQTMQDLDLQEVAGKWHSVAMAASDISLLDSESAPLRVYIEKLRPTPEDNLEIILREGENKGCAEKKIFAEKTESPAEFKINYLDEDTVFALDTDYKNYLFLCMKNAATPGQSLVCQYLARTQMVDEEIMEKFRRALQPLPGRVQIVPDLTRMAERCRI

SEQ ID NO: 8 - protein, 163 aa, Equus caballus
TDIPQTMQDLDLQEVAGRWHSVAMVASDISLLDSESVPLRVYVEELRPTPEGNLEIILREGANHACVERNIVAQKTEDPAVFTVNYQGERKISVLDTDYAHYMFFCVGPPLPSAEHGMVCQYLARTQKVDEEVMEKFSRALQPLPGRVQIVQDPSGGQERCGF

SEQ ID NO: 9 - protein, 162 aa, Equus asinus
TNIPQTMQDLDLQEVAGKWHSVAMAASDISLLDSEEAPLRVYIEKLRPTPEDNLEIILREGENKGCAEKKIFAEKTESPAEFKINYLDEDTVFALDSDYKNYLFLCMKNAATPGQSLVCQYLARTQMVDEEIMEKFRRALQPLPGRVQIVPDLTRMAERCRI

SEQ ID NO: 10 - protein, 163 aa, Equus asinus; identical to SEQ ID NO: 8

SEQ ID NO: 11 - protein, 162 aa, Rangifer tarandus
IIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 12 - protein, 123 aa, Bos taurus
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 13 - protein, 199 aa, Bos taurus
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 14 - protein, 199 aa, Ovis aries
RPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 15 - protein, 199 aa, Capra hircus
RPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 16 - protein, 199 aa, Bubalus bubalis
RPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 17 - protein, 197 aa, Equus caballus
RPKLPHRQPEIIQNEQDSREKVLKERKFPSFALEYINELNRQRELLKEKQKDEHKEYLIEDPEQQESSSTSSSEEVVPINTEQKRIPREDMLYQHTLEQLRRLSKYNQLQLQAIHAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 18 - protein, 215 aa, Camelus dromedarius
RPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 19 - protein, 170 aa, Homo sapiens
RPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 20 - protein, 207 aa, Bos taurus
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 21 - protein, 208 aa, Ovis aries
KHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 22 - protein, 208 aa, Capra hircus
KHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 23 - protein, 207 aa, Bubalus bubalis
KHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 24 - protein, 199 aa, Equus caballus
KHNMEHRSSSEDSVNISQEKFKQEKYVVIPTSKESICSTSCEEATRNINEMESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 25 - protein, 178 aa, Camelus dromedarius
KHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 26 - protein, 689 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 27 - protein, 691 aa, Homo sapiens
GRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 28 - protein, 17 aa, Homo sapiens
WNLLRQAQEKFGKDKSP

SEQ ID NO: 29 - protein, 17 aa, Bos taurus
WKLLSKAQEKFGKNKSR

SEQ ID NO: 30 - protein, 11 aa, Homo sapiens
GRRRSVQWCAV

SEQ ID NO: 31 - protein, 11 aa, Bos taurus
APRKNVRWCTI

SEQ ID NO: 32 - protein, 25 aa, Homo sapiens
TKCFQWQRNMRKVRGPPVSCIKRDS

SEQ ID NO: 33 - protein, 25 aa, Bos taurus
FKCRRWQWRMKKLGAPSITCVRRAF

SEQ ID NO: 34 - protein, 410 aa, Komagataella phaffii
MIFDGTTMSIAIGLLSTLGIGAEAKVHSAKIHKHPVSETLKEANFGQYVSALEHKYVSLFNEQNALSKSNFMSQQDGFAVEASHDAPLTNYLNAQYFTEVSLGTPPQSFKVILDTGSSNLWVPSKDCGSLACFLHAKYDHDESSTYKKNGSSFEIRYGSGSMEGYVSQDVLQIGDLTIPKVDFAEATSEPGLAFAFGKFDGILGLAYDSISVNKIVPPIYKALELDLLDEPKFAFYLGDTDKDESDGGLATFGGVDKSKYEGKITWLPVRRKAYWEVSFDGVGLGSEYAELQKTGAAIDTGTSLIALPSGLAEILNAEIGATKGWSGQYAVDCDTRDSLPDLTLTFAGYNFTITPYDYTLEVSGSCISAFTPMDFPEPIGPLAIIGDSFLRKYYSVYDLGKDAVGLAKSI

SEQ ID NO: 35 - protein, 410 aa, Komagataella pastoris
MMFDGTTMSIAIGLLSTLGIGVEANVHSAKIHKHPVSETLKEANFGQYVSALEHKYVSLFNEQNTLSKSNFVSQQDGFAIEASHDAPLTNYLNAQYFTEVSLGTPPQSFKVILDTGSSNLWVPSKDCGSLACFLHAKYDHDESSTYKKNGSSFEIRYGSGSMEGYVSQDVLQIGDLTIPKVDFAEATSEPGLAFAFGKFDGILGLAYDSISVNKIVPPIYKALELDLLDEPKFAFYLGDTDKDESDGGLATFGGVDKSKYEGKITWLPVRRKAYWEVSFDGVGLGSEYAELQKTGAAIDTGTSLIALPSGLAEILNAEIGATKGWSGQYAVDCDTRDSLPDLTLTFAGYNFTITPYDYTLEVSGSCISAFTPMDFPEPIGPLAIIGDSFLRKYYSVYDLGNDAVGLAKSI

SEQ ID NO: 36 - DNA, 762 bp, synthetic construct
  misc_feature 1..762: note = Synthetic construct
  source 1..762: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 37 - DNA, 762 bp, synthetic construct
  misc_feature 1..762: note = Synthetic construct
  source 1..762: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 38 - DNA, 4313 bp, synthetic construct
  misc_feature 1..4313: note = Synthetic construct
  source 1..4313: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 39 - DNA, 4313 bp, synthetic construct
  misc_feature 1..4313: note = Synthetic construct
  source 1..4313: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 40 - DNA, 9525 bp, synthetic construct
  misc_feature 1..9525: note = Synthetic construct
  source 1..9525: mol_type = other DNA
gcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctatgcggccgccaccaagtatcacagttggattaattagatatgaaaactaattgtttatttacaataagacgcagatagcccctttcgttcagaatagtcatttcatcatatttcctgatatatatgtctacattagtcttcaattgaactctgtcacagtggccgagtggttaaggcgatgccctgctattctttgaaagtagttaaggcattgggttctacctgcgcaggttcgaatcctgtctgtgacgagcatcatgcgaagcttcaagttttagagctagaaatagcaagttaaaataaggctagtccgttatcaacttgaaaaagtggcaccgagtcggtggtgctttttttgttttttatgtctcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaatcaacatctttggataatatcagaatgagaaagaacagatacgcagtacgttttttggtgagctctttgcacttctttagttctttccatcaatatcagttttttaaacttttaggactaaaagtgatgtttaacttcaaaatgtttaaaattttgttcttcccgacgttcattaagaatactaatacactttaataattattttaatattttgttctaaataatgacttttaattaaaaaagataaaatataaaaacatcataataactcaccagaggttaagaacaaaaaaacaaattagatatctgctaatccaatatagttaaatcaatctttccttggtataatgggtatattacatatatttcaaggaccgacactcctaccaaatatctaaaatttaccatattaacataacatgtatataaacgtcaaatcataatcagcactagatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggc

SEQ ID NO: 41 - DNA, 4893 bp, synthetic construct
  misc_feature 1..4893: note = Synthetic construct
  source 1..4893: mol_type = other DNA
gatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgg

SEQ ID NO: 42 - DNA, 444 bp, synthetic construct
  misc_feature 1..444: note = Synthetic construct
  source 1..444: mol_type = other DNA
aagtatcacagttggattaattagatatgaaaactaattgtttatttacaataagacgcagatagcccctttcgttcagaatagtcatttcatcatatttcctgatatatatgtctacattagtcttcaattgaactctgtcacagtggccgagtggttaaggcgatgccctgctattctttgaaagtagttaaggcattgggttctacctgcgcaggttcgaatcctgtctgtgacgagcatcatgcgaagcttcaagttttagagctagaaatagcaagttaaaataaggctagtccgttatcaacttgaaaaagtggcaccgagtcggtggtgctttttttgttttttatgtctcggtggagctccagcttttgttccctttagtgagggttaattgcgcgcttggcgtaatcatggtcatagctgtttcctgtgtgaaat

SEQ ID NO: 43 - DNA, 2033 bp, synthetic construct
  misc_feature 1..2033: note = Synthetic construct
  source 1..2033: mol_type = other DNA
gaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagattgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttc

SEQ ID NO: 44 - DNA, 86 bp, synthetic construct
  misc_feature 1..86: note = Synthetic construct
  source 1..86: mol_type = other DNA
atctaaaccattttacattctagagttccatttctcaattactgataatcaatttaaagaaacatccaaagacgaaaggttgaatg

SEQ ID NO: 45 - DNA, 87 bp, synthetic construct
  misc_feature 1..87: note = Synthetic construct
  source 1..87: mol_type = other DNA
tacatcttcactacctgataagtaaccaaataagttcagctgagcaacttttattcttgcccgcacaaacgaaggtttcacttaatc

SEQ ID NO: 46 - DNA, 2091 bp, synthetic construct
  misc_feature 1..2091: note = Synthetic construct
  source 1..2091: mol_type = other DNA
atctaaaccattttacattctagagttccatttctcaattactgataatcaatttaaagaaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagattgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcgggcaagaataaaagttgctcagctgaacttatttggttacttatcaggtagtgaagatgta

(19) United States
(12) Patent Application Publication
(10) Pub. No.: US 2026/0152753 A1
(43) Pub. Date: Jun. 4, 2026

(54) RECOMBINANT MILK POLYPEPTIDE COMPOSITIONS FREE OF ASPARTYL PROTEASE ACTIVITY

(71) Applicant: DSM IP ASSETS B.V., Heerlen (NL)

(72) Inventors: Robertus Antonius Mijndert VAN DER HOEVEN, Echt (NL); Skelte-Gerald ANEMA, Auckland (NZ); Bernard MEIJRINK, Echt (NL); Sheelagh Ann HEWITT, Auckland (NZ); Alan David WELMAN, Auckland (NZ); Jeremy Paul HILL, Auckland (NZ)

(21) Appl. No.: 18/707,670

(22) PCT Filed: Nov. 10, 2022
(86) PCT No.: PCT/EP2022/081513

§ 371 Date: May 6, 2024

(30) Foreign Application Priority Data
Nov. 10, 2021 (AU) 2021903588

(51) Int. Cl. C12N 15/81; A23C 11/08; A23J 1/18; C12N 9/22; C12N 15/11; C12P 21/00

(52) U.S. Cl. CPC C12N 15/815; A23C 11/08; A23J 1/18; C12N 9/226; C12N 15/11; C12P 21/005; C12N 2310/20; C12N 2800/102

(57) ABSTRACT

The present invention relates to food products comprising recombinant polypeptide compositions that are substantially free of aspartyl protease-like activity. The invention also relates to compositions comprising recombinant milk polypeptides and that are substantially free of aspartyl protease-like activity; and methods and tools for manufacture of such compositions.

### FIELD OF THE INVENTION

The present invention relates to food products comprising recombinant polypeptide compositions that are substantially free of aspartyl protease-like activity. The invention also relates to compositions comprising recombinant milk polypeptides and that are substantially free of aspartyl protease-like activity; and methods and tools for manufacture of such compositions.

### BACKGROUND TO THE INVENTION

With the increasing popularity of plant-based foods, for the same volume of consumption, consumers will receive less nutrition, particularly protein and amino acid nutrition, as is found in similar products that contain milk protein or other animal proteins. Consumers wanting to make a plant-based choice in preference to products containing animal-proteins, will find that they often have inferior sensory attributes, such as unfavourable flavour, odour and/or colour. Food manufacturers making such plant-based foods are also losing out on the functional benefits of milk protein ingredients such as thickening, gelling, texture modification, heat stability and foaming attributes.

Biosynthetically—manufactured animal proteins, including those from a milk source, can be used in plant-based and animal-based foods to address the abovementioned deficit in nutrition and unfavourable functional and sensory properties associated with plant-based proteins. However, it is also important to ensure that the advantages imparted by these fermentatively-produced food ingredients, when used in particular food applications, do not impart other undesirable functional and/or organoleptic characteristics to the food product.

It is an object of the present invention to provide improved or alternative compositions comprising recombinant milk polypeptides and food products comprising recombinant polypeptides, and/or to at least provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In a first aspect the invention relates to a food product comprising a composition comprising one or more recombinant polypeptides, wherein the composition is (substantially) free of aspartyl protease-like activity. Preferably wherein the recombinant polypeptide comprise or are β-lactoglobulin, such as bovine β-lactoglobulin.

In various embodiments the food product comprises casein. In various embodiments the food product comprises κ-casein.

In various embodiments the food product may comprise any composition of the invention or described herein.

In another aspect, the invention relates to a method for preparing a food product, the method comprising
a) providing a composition comprising one or more recombinant polypeptides, wherein the composition is substantially free of aspartyl protease-like activity, and
b) mixing the composition with one or more additional ingredients to produce the food product, preferably wherein at least one ingredient comprises casein.

In various embodiments the method comprises mixing the composition with one or more additional ingredients to produce the food product, preferably wherein at least one ingredient comprises κ-casein.

In one aspect the invention provides the use of a composition comprising one or more recombinant polypeptides, wherein the composition is substantially free of aspartyl protease-like activity to produce a food product, preferably wherein the food product comprises casein.

In various embodiments the food product comprises κ-casein.

In various embodiments the composition may be any composition of the invention or described herein.

In various embodiments the composition may comprise one or more recombinant animal polypeptides or plant polypeptides. In various embodiments the composition may comprise one or more recombinant animal proteins or plant proteins. In various embodiments the composition may comprise one or more recombinant milk polypeptides. In some embodiments, the recombinant milk polypeptides may be recombinant dairy polypeptides, preferably β-lactoglobulin, such as bovine β-lactoglobulin.

In various embodiments the method may comprise providing any composition of the invention or described herein.

In a further aspect, the invention provides a composition comprising one or more recombinant milk polypeptides, wherein the composition is substantially free of aspartyl protease-like activity.

In another aspect, the invention relates to a host cell for producing one or more recombinant milk polypeptides, wherein
a) the species of the host cell is selected from Pichia pastoris, Kluyveromyces lactis, Aspergillus niger and Saccharomyces cerevisiae,
b) the host cell lacks an operative pep4 gene, or has been modified to produce less PEP4 protein than a wild type control cell, and
c) the host cell expresses one or more milk polypeptides, preferably β-lactoglobulin, such as bovine β-lactoglobulin.

In various embodiments the host cell genome may comprise one or more integrated sequences that encode one or more milk polypeptides, preferably β-lactoglobulin, such as bovine β-lactoglobulin.

In various embodiments the host cell comprises a polynucleotide encoding one or more milk polypeptides, preferably β-lactoglobulin, such as bovine β-lactoglobulin.

In various embodiments the polynucleotide encodes
a) a signal sequence,
b) a leader sequence,
c) a milk polypeptide, and
d) a processing site selected from KR, KREA, KREAEA and KREAEAM located between the leader sequence and the sequence encoding the milk polypeptide.

In a further aspect, the invention relates to a method for producing one or more recombinant milk polypeptides comprising
a) culturing a host cell of the invention in a culture medium under conditions sufficient to allow for expression of the one or more recombinant milk polypeptides, and
b) isolating the one or more recombinant milk polypeptides from the culture medium.

In another aspect the invention relates to a composition comprising one or more recombinant milk polypeptides, wherein the composition is produced according to a method described herein, preferably wherein the recombinant milk polypeptide is β-lactoglobulin, such as bovine β-lactoglobulin.

In one aspect, the invention relates to a kit for replacing a pep4 gene in a host cell with a sequence encoding one or more milk polypeptides, the kit comprising
a) a polynucleotide expression vector encoding
   i) a marker to select for transformed host cells,
   ii) a sequence that enables autonomous replication of the polynucleotide expression vector in the host cell,
   iii) a first promoter sequence,
   iv) a Cas9 protein,
   v) a terminator sequence,
   vi) a second promoter sequence, and
   vii) a guide RNA targeting the pep4 gene; and
b) a polynucleotide encoding
   i) 5′ and 3′ integration sequences,
   ii) a promoter sequence,
   iii) a leader sequence,
   iv) a milk polypeptide;
   v) a processing site selected from KR, KREA, KREAEA and KREAEAM located between the leader sequence and the milk polypeptide; and
   vi) a terminator sequence.

In various embodiments the polynucleotide expression vector or the polynucleotide may encode a wild type milk polypeptide. In various embodiments the polynucleotide expression vector or the polynucleotide may encode a wild type, mature milk polypeptide. In various embodiments the wild type milk protein is a bovine milk polypeptide.

In various embodiments the polynucleotide expression vector or the polynucleotide may encode two or more, three or more, four or more, five or more or six or more milk polypeptides.

In various embodiments the polynucleotide expression vector or the polynucleotide may encode a β-lactoglobulin polypeptide, an α-lactalbumin polypeptide, a casein polypeptide or a lactoferrin polypeptide.

In various embodiments the polynucleotide expression vector or the polynucleotide may encode
a) a β-lactoglobulin polypeptide having at least 75%, 80%, 85%, 90%, 95% or 99% sequence identity, or having 100% sequence identity to one of SEQ ID Nos:1-11;
b) an α-lactalbumin polypeptide having at least 75%, 80%, 85%, 90%, 95% or 99% sequence identity, or having 100% sequence identity to SEQ ID No:12,
c) a casein polypeptide having at least 75%, 80%, 85%, 90%, 95% or 99% sequence identity, or having 100% sequence identity to any one of SEQ ID Nos: 13-25; or
d) a lactoferrin polypeptide having at least 75%, 80%, 85%, 90%, 95% or 99% sequence identity, or having 100% sequence identity to any one of SEQ ID Nos:28-33.

In various embodiments the processing site may be selected from KR, KREA, and KREAEAM. In other embodiments the processing site may be selected from KREA, KREAEA and KREAEAM.

The following embodiments may relate to any of the above aspects.

In various embodiments the recombinant milk polypeptides have at least about 70%, 75%, 80%, 85%, 90%, 95%, or 99% sequence identity or have 100% sequence identity to a wild type, mature milk protein. In various embodiments the wild type, mature milk protein is a wild type, mature β-lactoglobulin, α-lactalbumin, casein or lactoferrin protein. In various embodiments the wild type, mature milk protein is a bovine protein.

In some embodiments the recombinant milk polypeptides are recombinant dairy polypeptides.

In various embodiments the one or more recombinant milk polypeptides comprises one or more β-lactoglobulin proteins, α-lactalbumin proteins, casein proteins, lactoferrin proteins, or any combination of any two or more thereof.

In various embodiments, the wild type, mature milk protein has a sequence selected from any one of SEQ ID Nos: 1-27.

In various embodiments the recombinant milk polypeptides have at least about 70%, 75%, 80%, 85%, 90%, 95%, or 99% sequence identity or have 100% sequence identity to any one of SEQ ID Nos:1-33.

In various embodiments the recombinant milk polypeptide is a recombinant milk protein. In various embodiments the milk protein may be selected from a mature protein or a full length protein.

In various embodiments the wild type, mature milk protein is
a) a β-lactoglobulin protein having a sequence selected from any one of SEQ ID Nos: 1-11;
b) an α-lactalbumin protein having a sequence of SEQ ID No: 12;
c) a casein protein having a sequence selected from any one of SEQ ID Nos: 13-25; or
d) a lactoferrin protein having a sequence of SEQ ID No: 26 or 27.

In other embodiments the recombinant milk polypeptide may be a peptide fragment of a milk protein. In various embodiments the polypeptide comprises from about 1 to about 40, about 1 to about 35, about 1 to about 30, about 1 to about 25, about 1 to about 20, about 1 to about 15 or about 1 to about 12 contiguous amino acids of a wild type milk protein sequence. In various embodiments the peptide fragment is lactoferrampin or lactoferricin or LF1-11.

In various embodiments the one or more recombinant milk polypeptides have at least 70%, 80%, 90%, 95% or 99% sequence identity, or 100% sequence identity, to a polypeptide having a sequence selected from any one of SEQ ID Nos: 28 to 33.

In various embodiments the recombinant milk polypeptide may be a glycopeptide or glycoprotein.

In various embodiments, when the composition is added to a skim milk composition comprising 10% by weight total solids to a concentration of the one or more recombinant polypeptides of about 1% by weight and the skim milk composition is heated at 80° C. for 30 minutes then held at 5° C. for 6 hours,
a) no degradation of κ-casein is observed, and/or
b) no production of para-κ-casein is observed.

In various embodiments, when the composition is added to a skim milk composition comprising 10% by weight total solids to a concentration of the one or more recombinant polypeptides of about 1% by weight and incubated at ambient temperature for 24 hours, less than about 45%, 40%, 35%, 30%, 25%, 20%, 15%, 10%, 5% or 1%, or 0% of κ-casein in the skim milk composition is degraded to form para-κ-casein.

In various embodiments, when the composition is added to a skim milk composition comprising 10% by weight total solids to a concentration of the one or more recombinant proteins of about 1% by weight and the skim milk composition is held at ambient temperature for 15 minutes then heated at 140° C., the composition has not coagulated after about 14 minutes.

In various embodiments, when the composition is added to a skim milk composition comprising 10% by weight total solids to a concentration of the one or more recombinant proteins of about 1% by weight, and the skim milk composition is heated at 40° C. for 12 hours, the skim milk composition does not reach gelation point.

In various embodiments the composition is substantially free of aspartyl protease
a) consisting of an amino acid sequence of SEQ ID no. 34 or 35, or
b) having at least 75%, 80%, 85%, 90%, 95% or 99% sequence identity to SEQ ID Nos: 34 or 35.

In various embodiments the food product may comprise an additional source of protein. In various embodiments the additional source of protein is a non-dairy source. In various embodiments the non-dairy source of protein may comprise a plant source, algal protein, mycoprotein, or a combination thereof.

In various embodiments the plant source may comprise one or more legumes, grains, seeds, nuts, tubers, or any combination of any two or more thereof.

In various embodiments
a) the legumes may comprise soy, pea, lentil, chickpea, peanut, bean or any combination of any two or more thereof;
b) the grains may comprise wheat, rice, oat, corn or any combination of any two or more thereof;
c) the seeds may comprise canola, flaxseed (linseed), hemp, sunflower, quinoa, chia, or any combination of any two or more thereof;
d) the nuts may comprise almond, cashew, walnut or any combination of any two or more thereof; and/or
e) the tuber may comprise potato.

In various embodiments the food product is suitable for those on a vegan diet.

In various embodiments the food product comprises milk-derived casein, optionally dairy milk-derived casein, one or more recombinant casein polypeptides, or a combination thereof.

In various embodiments the food product comprises milk-derived κ-casein, optionally dairy milk-derived κ-casein, recombinant κ-casein, or a combination thereof.

In various embodiments the food product or the one or more additional ingredients may comprise whole milk, skim milk, a milk protein concentrate (MPC), a milk protein isolate (MPI), micellar casein, or a combination of any two or more thereof.

In various embodiments the food product is selected from the group comprising a fermented food, a yoghurt, a soup, a sauce, a bar, a gel, a nutritional formulation, a beverage, a beverage whitener, a cheese, a dairy tofu and a dessert.

In various embodiments the cheese may be fresh cheese. In various embodiments the cheese may be processed cheese, Petit-Suisse, cottage cheese, or quark.

In various embodiments the nutritional formulation is selected from the group comprising an infant formula, a follow-on formula, a toddler milk, a growing up formula, a maternal formula, a food for active lifestyles, a medical food and a supplement.

In various embodiments the beverage is selected from the group comprising a dairy beverage, a sports beverage, a smoothie, a protein fortified fruit or vegetable juice, a drinking yoghurt, an acid protein fortified beverage, liquid coffee, liquid tea, and a liquid beverage whitener.

The term “mature” as used herein with reference to milk proteins refers to the protein, or amino acid sequence of the protein, after cleavage of the signal sequence. Examples of mature milk protein sequences are provided in Table 1 herein. The term “full length” as used herein with reference to milk proteins refers to the protein, or amino acid sequence of the protein, comprising the signal sequence.

The term “polypeptide” as used herein refers to any polymer of amino acids residues of any length. In various embodiments the polypeptide is a protein or protein fragment, including any of the proteins described herein. In various embodiments the polypeptide is from about 1 to about 200, 1 to about 100, 1 to about 50, 1 to about 40, 1 to about 30, 1 to about 20, from about 5 to about 40, 5 to about 30, 5 to about 20, about 10 to about 50, about 10 to about 40, about 10 to about 30, about 10 to about 20 amino acids in length. The polypeptide may be a fragment of a wild type protein, such as a wild type (full length or mature) milk protein. The polypeptide may have a sequence of contiguous amino acid resides from the wild type milk protein sequence. In various embodiments, the polypeptides described herein may comprise one or more post-translational modifications, including glycosylation and or phosphorylation at one or more residues.

The term “milk” as used herein refers to mammalian milk from any species. In various embodiments the milk may be obtained from any species including bovine, ovine, caprine, equine, reindeer, buffalo, human and camel.

The term “dairy milk” as used herein refers to non-human, mammalian milk that may be consumed by humans. In various embodiments the milk may be bovine, ovine, caprine, equine, reindeer, buffalo, camel milk.

The term “milk polypeptide” as used herein refers to any protein or peptide present in mammalian milk from any species. In various embodiments the milk polypeptide has at least 70%, 75%, 80%, 85%, 90%, 95%, 96%, 97%, 98% or at least about 99% sequence identity to a wild type (native) milk protein, protein fragment or peptide.

The term “dairy polypeptide” as used herein refers to protein or peptide present in non-human mammalian milk from any species. In various embodiments the dairy polypeptide has at least 70%, 75%, 80%, 85%, 90%, 95%, 96%, 97%, 98% or at least about 99% sequence identity to a wild type (native) dairy protein, protein fragment or peptide.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The terms “variant” as used herein may include proteins comprising an amino acid sequence having at least about 70%, 75%, 80%, 85%, 90%, 95%, 96%, 97%, 98% or at least about 99% sequence identity to the sequence of a wild type (native) milk protein (either full length or mature protein lacking a signal sequence, but preferably the mature sequence), but particularly any native bovine, ovine, caprine, buffalo, equine, donkey or reindeer sequence, including any sequence of SEQ ID Nos:1 to 27. In some embodiments the variant refers to a natural variant, for example, one or more of variants A, B and C of bovine β-lactoglobulin. In some embodiments the amino acid sequence of such variants may comprise truncations or elongations at the N-terminus and/or the C-terminus relative to the wild type sequence, for example, elongations or truncations of from about 1 to about 20 amino acids. In some embodiments, variants may contain from 1 to 20 amino acid insertions, deletions, and/or substitutions (collectively) with respect to the wild type sequence. In some embodiments the variants may comprise one or more post-translational modifications that differ to a wild type milk protein, including glycosylation and or phosphorylation at one or more residues.

The term “wild type” as used herein with reference to proteins or polynucleotides refers to a protein or polynucleotide having an amino acid or nucleotide sequences that is the same as that expressed naturally in any species. This term includes all naturally occurring variants of a particular protein, for example, all naturally occurring variants of β-lactoglobulin, α-lactalbumin, casein proteins or lactoferrin. Furthermore, this term includes both full length and mature milk proteins and polynucleotides that encode wild type full length and mature milk proteins. The term is generally synonymous with the term “native”.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprised” are to be interpreted in a similar manner.

As used herein the term “and/or” means “and” or “or”, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

To those skilled in the art to which the invention relates, many changes in construction and differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

The invention will now be described by way of example only and with reference to the drawings in which:

FIG. 1 provides plasmid maps of the following expression constructs: (A) pLGAAOX-005 for expression of β-lactoglobulin variant A; and (B) pLGBAOX-005 for expression of the β-lactoglobulin variant B.

FIG. 2 provides a plasmid map of the expression construct pCASPP-05.

### DETAILED DESCRIPTION OF THE INVENTION

The present invention relates to food products comprising recombinant polypeptide compositions that are substantially free of aspartyl protease-like activity.

The invention also relates to compositions comprising recombinant milk polypeptides and that are substantially free of aspartyl protease-like activity and methods of producing the compositions.

The present inventors have shown that the recombinant protein compositions described herein are useful as functional ingredients in protein-containing food products including fermented foods, yoghurts, drinking yoghurts, soups, sauces, bars, gels, nutritional formulations, beverages, beverage whiteners, milk “tofu” and desserts.

The compositions confer certain advantages when used as functional ingredients in foods due to the avoidance of undesirable gelling caused by unwanted proteolysis of casein, particularly κ-casein, in foods. The present inventors have determined that unwanted proteolysis of casein in food products may be induced by aspartyl proteases present in the functional ingredient that have been produced by host cells used for recombinant production of proteins. The present inventors have demonstrated that compositions that are substantially free of aspartyl protease-like activity can be produced that avoid this undesirable gelling. Advantages conferred by the compositions described herein include improved heat stability, improved flavour and/or the avoidance of undesirable flavours, reduced sedimentation in beverages and enhanced storage stability.

### 1. Recombinant Polypeptide Compositions

The compositions described herein may comprise any recombinant polypeptides, but preferably animal or plant polypeptides. In various embodiments the recombinant polypeptides may consist of, or have at least 70% sequence identity to any wild type protein, peptide or peptide fragment from any species. In various embodiments the compositions described herein comprise one or more recombinant proteins having at least 70% sequence identity to a bovine, ovine, caprine, equine, reindeer, buffalo, human or camel wild type protein.

The recombinant milk polypeptides in the compositions described herein may consist of, or have at least 70% sequence identity to any wild type, mature milk protein or full length, mature milk protein that is naturally present in mammalian milk from any species including bovine, ovine, caprine, equine, reindeer, buffalo, human and camel. The milk proteins may include any whey protein, including β-lactoglobulin and/or α-lactalbumin, any casein protein, including αs1-casein, αs2-casein, beta casein, para k-casein and k-casein, lactoferrin and variants thereof. This can also include the full length and mature milk proteins lacking a signal sequence. In some embodiments the recombinant milk polypeptides comprise a peptide fragment of a wild type, milk protein.

In various embodiments the composition may comprise, or consist of, two or more, three or more, four or more, five or more or six or more recombinant polypeptides, preferably proteins. In some embodiments the two or more recombinant polypeptides have been produced in the same host cell or in one or more separate host cells. In various embodiments the two or more polypeptides in the composition interact to form macro-molecular structures, for example micelles. In various embodiments the two or more recombinant polypeptides comprise two or more milk polypeptides selected from αs1-casein, αs2-casein, beta casein, para k-casein, or any combination of any two or more thereof.

B-lactoglobulin is the major whey protein in the milk of many mammals. In bovine milk it accounts for approximately 10-15% of total milk proteins and about 50-54% of whey protein. Bovine β-lactoglobulin is expressed as a precursor protein comprising a 16 amino acid N-terminal signal peptide, which is cleaved to form a mature 162 amino acid protein.

There are two primary variants of bovine β-lactoglobulin—variants A and B. Sequences for both the mature and precursor forms of bovine β-lactoglobulin variants A and B, and β-lactoglobulin sequences from other species are presented in Table 1.

In various embodiments the one or more recombinant milk proteins may comprise one or more proteins having an amino acid sequence having 100% identity to a native (wild type) β-lactoglobulin that is derived from any species, for example, any of SEQ ID Nos 1-11.

In various embodiments the one or more recombinant milk proteins comprise one or more variants of a bovine β-lactoglobulin variant A, B or C. In some embodiments the one or more recombinant milk proteins have a sequence having at least 70% sequence identity to one or more variants of a bovine β-lactoglobulin variant A, B or C.

α-lactalbumin is the second major protein component of bovine whey and accounts for around 20% of total whey proteins. The α-lactalbumin protein is expressed as a precursor protein comprising a 16 amino acid N-terminal signal peptide, which is cleaved to form a mature 123 amino acid protein. The most common variant of α-lactalbumin is the B variant, which is in Bos taurus cattle. The A and B variants are found in Bos indicus cattle and the A variant is also found at low frequency in some Bos taurus breeds from Italy and East Europe. The variation is a Glu at position 10 in the A variant and a Arg at position 10 in the B variant.

In the majority of mammalian species, α-lactalbumin regulates the production of lactose in the milk. α-lactalbumin acts as the regulatory unit and forms a heterodimer with β-1,4-galactosyltransferase (beta4Gal-T1) to form lactose synthase. This enables lactose synthase to produce lactose by transferring galactose moieties to glucose.

Caseins are phosphoproteins found in mammalian milk. Caseins comprise about 80% of all proteins in bovine milk, but the proportion of casein relative to total protein varies considerably between species.

Casein proteins include αs-casein, β-casein and κ-casein. αs-casein may include αs1-casein and/or αs2-casein. In various embodiments the composition may comprise one or more recombinant αs-casein, β-casein or κ-casein. In various embodiments the recombinant αs-casein is a recombinant αs1-casein or αs2-casein.

Lactoferrin is a naturally occurring, monomeric, globular iron-binding glycoprotein that is produced by mammals. It has a known role as part of the immune system and in the first line of protection against pathogenic microbes. Lactoferrin comprises approximately 680 amino acids and has a molecular mass of approximately 80 kDa. Structurally, lactoferrin has two lobes (N- and C-terminal lobes), which share an internal homology of approximately 40% sequence identity with one another. Each lobe has two α/β domains divided by a cleft, that has an iron-binding site. The protein is also glycosylated at a number of sites.

In various embodiments the milk polypeptide comprises a lactoferrin fragment, for example, lactoferrampin, LF1-11 or lactoferricin. Lactoferrampin is an antimicrobial peptide located in the cationic N-terminal lobe of lactoferrin protein. It is found in both humans and cows. LF1-11 is LF1-11, is the N-terminal peptide of lactoferrin, comprised of the first eleven residues of the molecule. This peptide has been shown to be highly effective against some resistant bacteria. Lactoferricin is an amphipathic, cationic peptide which has been to shown to have anti-microbial properties. It is formed as a result of the digestion of lactoferrin, which is mediated by pepsin.

TABLE 1
Sequences for wild type milk proteins

| SEQ ID No. | Name |
|---|---|
| 1 | Bovine β-lactoglobulin variant A |
| 2 | Bovine β-lactoglobulin variant B |
| 3 | Bovine β-lactoglobulin variant C |
| 4 | Ovine β-lactoglobulin |
| 5 | Caprine β-lactoglobulin |
| 6 | Water buffalo β-lactoglobulin |
| 7 | Equine β-lactoglobulin variant I |
| 8 | Equine β-lactoglobulin variant II |
| 9 | Donkey β-lactoglobulin variant 1 |
| 10 | Donkey β-lactoglobulin variant 2 |
| 11 | Reindeer β-lactoglobulin |
| 12 | Bovine a-lactalbumin |
| 13 | Bovine Alpha-S1 Casein |
| 14 | Ovine Alpha S1 casein |
| 15 | Caprine Alpha S1 casein |
| 16 | Buffalo Alpha S1 Casein |
| 17 | Equine Alpha S1 Casein |
| 18 | Camel Alpha S1 casein |
| 19 | Human Alpha S1 Casein |
| 20 | Bovine Alpha-S2 Casein |
| 21 | Ovine Alpha S2 casein |
| 22 | Caprine Alpha S2 casein |
| 23 | Buffalo Alpha S2 Casein |
| 24 | Equine Alpha S2 Casein |
| 25 | Camel Alpha S2 casein |
| 26 | Bovine lactoferrin |
| 27 | Human lactoferrin |
| 28 | Human lactoferrampin |
| 29 | Bovine lactoferrampin |
| 30 | Human LF1-11 |
| 31 | Bovine LF1-11 |
| 32 | Human lactoferricin |
| 33 | Bovine lactoferricin |

### Properties of the Protein Compositions

During recombinant protein production, aspartyl proteases derived from the host cells are typically not entirely removed by purification.

Advantageously, the compositions described herein are substantially free of aspartyl protease-like activity.

Aspartyl proteases (also known as aspartic proteases) EC 3.423 are a catalytic type of protease enzyme that utilise an activated water molecule bound to one or more aspartate residues for catalysis of peptide substrates. Most aspartyl proteases have two highly conserved aspartates in the active site of the enzyme and are optimally active at acidic pH.

In various embodiments the compositions described herein may comprise an aspartyl protease having a sequence of SEQ ID No: 34 or 35.

The term “substantially free of aspartyl protease-like activity” as used herein refers to a composition that, when added to casein, in particular κ-casein, does not result in degradation of the casein and/or κ-casein. In some embodiments this term refers to a composition that, when added to casein, does not result in degradation of αs-casein, β-casein, κ-casein or any combination of any two or more thereof. In various embodiments, no degradation of κ-casein is observed and/or no production of para-κ-casein is observed when the compositions described herein are incubated with a substrate comprising κ-casein. In some embodiments, when the composition is added to κ-casein, less than about 45%, 40%, 35%, 30%, 25%, 20%, 15%, 10%, 5%, 1% or 0.5% of the κ-casein is degraded to para-κ-casein. In various embodiments, when the composition is added to a skim milk composition comprising 10% by weight total solids to a concentration of the one or more recombinant polypeptides of about 1% by weight and incubated at ambient temperature for 24 hours, less than about 45%, 40%, 35%, 30%, 25%, 20%, 15%, 10%, 5%, 1% or 0.5% of κ-casein in the skim milk composition is degraded to form para-κ-casein.

Methods for detecting aspartyl protease activity are known in the art and can be used with recombinant milk polypeptide compositions. One such method which can be used to measure aspartyl protease activity via κ-casein degradation is ‘lab-on-a-chip” sodium dodecyl sulphate poly acrylamide gel electrophoresis (SDS-PAGE) as described herein in the examples.

Briefly a recombinant polypeptide composition is added to a solution of skim milk solids, incubated for a period of time and then run on either an SDS gel or “lab on chip” to isolate and quantify the amount of κ-casein and its degradation product para-κ-casein. Detailed methods for this process can be found in “The use of “lab-on-a-chip” microfluidic SDS electrophoresis technology, S. G. Anema 2009, International Dairy Journal 19 (2009) 198-204”.

In some embodiments, the compositions of the invention have acceptable or improved nutritional value and/or protein quality compared with native milk proteins.

### 2. Recombinant Expression

The recombinant milk proteins in the compositions of the invention are produced by recombinant expression in a host cell. As used herein, a “host” or “host cell” denotes any protein production host selected or genetically modified to produce a desired product. Exemplary hosts include fungi, such as filamentous fungi, as well as bacteria, yeast, algae, plant, insect, and mammalian cells.

In various embodiments, the host cell is a yeast cell selected from the list consisting of Pichia pastoris (also known as Komagataella phaffii), Kluyveromyces lactis, Saccharomyces cerevisiae, Schizosaccharomyces pombe, Yarrowia lipolytica, Candida glabrata, Ashbya gossypii, Cyberlindnera jadinii, Pichia methanolica, Hansenula polymorpha, Candida boidinii, Arxula adeninivorans, Xanthophyllomyces dendrorhous, and Candida albicans species. In some embodiments, the yeast cell is a Saccharomycete.

In some embodiments, the host cell is a fungal cell selected from the list consisting of Aspergillus spp. and Trichoderma spp.

In various embodiments the host cell is selected from Pichia pastoris Kluyveromyces lactis, Aspergillus niger and Saccharomyces cerevisiae.

In some embodiments, the host cell may be a bacterial host cell such as Lactococcus lactis, Bacillus subtilis or Escherichia coli. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum and Synechocystis sp. 6803.

Host cells useful for producing the protein compositions described herein may be prepared by functional knockout or replacement of the endogenous pep4 gene in the host cell. Suitable methods for achieving functional knockout or gene replacement are well known in the art, and include CRISPR/CAS9 technology, mutation of the coding sequence to modify the amino acid sequence of the protein encoded by the gene to render it non functional, or by deletion and/or insertion of nucleotides into the coding sequence. Alternatively, the promoter expressing the pep4 gene can be replaced by another promoter with low regulated expression.

In one embodiment, the functional knockout/gene replacement of pep4 and insertion of an expression cassette for the one or more recombinant milk proteins may be accomplished simultaneously using CRISPR-CAS9 technology. An example is described herein in the examples. Briefly, CRISPR-CAS9 may be used to integrate a fragment containing the one or more recombinant milk proteins and deleting the pep4 gene in the host cell. An “all in one” expression vector may be used, which expresses both CAS9 and guide RNA (gRNA) targeting the pep4 gene sequence. In various embodiments the CAS9 gene and the pep4 gRNA are under the control of separate promoters. Preferably, the plasmid contains a selectable marker, such as an antibiotic selectable marker and an autonomously replicating sequence to select and maintain the plasmid in the cell after transformation to Pichia.

Host cells comprising genetic constructs, such as expression constructs, as disclosed herein may be used in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides disclosed herein. Such methods may involve the culture of host cells in an appropriate medium in conditions suitable for or conducive to expression of a polynucleotide or polypeptide disclosed herein. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

Expression of a target protein can be provided by an expression vector, a plasmid, a nucleic acid integrated into the host genome or other means. For example, in some embodiments the host cell comprises a polynucleotide, e.g. an expression vector that comprises a sequence encoding a β-lactoglobulin protein (x) and may additionally comprise one or more of (a) a promoter element, (b) a signal peptide sequence, (c) a leader sequence, (d) a processing site and (e) a terminator element.

The promoter (a) and leader sequence (c) may be, or may be derived from, any suitable yeast, fungal, bacterial or mammalian promoter or leader sequence.

In various embodiments the host cell comprises a polynucleotide, such as an expression vector, comprising a processing site located between a leader sequence and the sequence encoding a milk polypeptide. In various embodiments the processing site may be a KEX processing site. In various embodiments the processing site may have the sequence KREAEA, KR or KREAEAM.

Expression vectors that can be used for expression of a milk protein include those containing an expression cassette with elements (a), (b), (c), (d) and/or (e). In some embodiments, the signal peptide sequence (b) need not be included in the vector. In some cases, the vector comprises a polynucleotide encoding a milk protein sequence as exemplified in SEQ ID NOs: 1 to 33. In general, the expression cassette is designed to mediate the transcription of the transgene when integrated into the genome of a cognate host microorganism or when present on a plasmid or other replicating vector maintained in a host cell.

To aid in the amplification of the vector prior to transformation into the host microorganism, a replication origin (f) may be contained in the vector. To aid in the selection of microorganism stably transformed with the expression vector, the vector may also include a selection marker (g). The expression vector may also contain a restriction enzyme site (h) that allows for linearization of the expression vector prior to transformation into the host microorganism to facilitate the expression vectors stable integration into the host genome. The expression vector may contain integration sequences (i) homologous to genomic sequences of the host cell that enable or aid integration of a fragment of the expression vector or the entire expression vector into the genome of the host cell. In some embodiments the expression vector may contain any subset of the elements (a) to (i). Other expression elements and vector element known to one of skill in the art can be used in combination or substituted for the elements described herein. For example, because many microorganisms are capable of expressing multiple gene products from a polycistronic mRNA, multiple polypeptides can be expressed under the control of a single regulatory region for those microorganisms, if desired. These might include one or more β-lactoglobulin proteins, one or more different milk proteins (for example, one or more caseins, α-lactalbumin or lactoferrin proteins), and/or one or more non-milk proteins.

Gram positive bacteria (such as Lactococcus lactis and Bacillus subtilis) may be used to secrete target proteins into the media, and gram-negative bacteria (such as Escherichia coli) may be used to secrete target proteins into periplasm or into the media. In some embodiments, the bacterially-expressed proteins expressed may not have any post-translational modifications (PTMs), which means they are not glycosylated and/or may not be phosphorylated.

Recombinant milk proteins may be expressed and produced in L. lactis both in a nisin-inducible expression system (regulated by PnisA promoter), lactate-inducible expression system (regulated by P170 promoter) or other similar inducible systems, as well as a constitutively expressed system (regulated by P secA promoter), wherein both are in a food-grade selection strain, such as NZ3900 using vector pNZ8149 (lacF gene supplementation/rescue principle). The secretion of functional proteins may be enabled by the signal peptide of Usp45 (SP(usp45)), the major Sec-dependent protein secreted by L. lactis.

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3rd edition), Cold Spring Harbor Laboratory, Cold Spring Harbor Laboratory Press, or F. Ausubel et al, eds., “Current protocols in molecular biology”, Green Publishing and Wiley Interscience, New York (1987).

Compositions comprising the recombinant milk polypeptide may be produced by culturing a host cell expressing the polypeptide using standard methods well known in the art. The cell culture biomass may then be centrifuged to remove solid matter and the light phase subjected to a series of filtration and chromatography steps to remove the aspartyl protease from the recombinant milk polypeptide.

In one exemplary embodiment the light phase may be subjected to Polish filtration followed by sterile filtration. After desalting and dilution, the composition may be subjected to chromatography followed by ultrafiltration with diafiltration.

The above methods may be used to produce compositions for use in the invention comprising any recombinant polypeptide.

### 3. Food Products

The invention also relates to food products comprising a composition comprising a plurality of recombinant milk proteins described herein, and methods of producing such food products.

The composition may be mixed with one or more additional ingredients to produce a food product. In various embodiments, the method may comprise mixing the composition with one or more additional ingredients to produce a food product. The food product may be any edible consumer product which is able to carry protein.

In various embodiments, the food product may comprise at least about 1%, 1.5%, 2%, or 2.5% total protein by weight. In various embodiments, the food product may comprise from about 1 to about 50%, about 1 to about 45%, about 1 to about 40%, 1 to about 35%, about 1 to about 30%, or about 1% to about 25% total protein by weight, and useful ranges may be selected from between any of these values (for example, from about 1% to about 20%, or about 1% to about 16%, 1% to about 15%, 1% to about 14%, or about 1% to about 12%, or about 1% to about 10%, or about 2% to about 20%, or about 2% to about 16%, 2% to about 15%, 2% to about 14%, or about 2% to about 12%, or about 2% to about 10%, about 4% to about 20%, or about 4% to about 16%, 4% to about 15%, 4% to about 14%, or about 4% to about 12%, or about 4% to about 10%, about 5% to about 20%, or about 5% to about 16%, 5% to about 15%, 5% to about 14%, or about 5% to about 12%, or about 5% to about 10%).

In various embodiments the food product may be a fermented food, a yoghurt, a soup, a sauce, a bar, a gel, a nutritional formulation, a beverage, a beverage whitener, a cheese, a dairy tofu or a dessert:

In various embodiments the food product is free of animal-derived ingredients. In various embodiments the food product is considered suitable for those on a vegan diet. The food product may comprise one or more additional sources of protein, including the examples described herein. In various embodiments the additional source of protein is a non-dairy source of protein, such as a plant protein described herein or algal protein or mycoprotein.

Nutritional formulations may include infant formulas, follow-on formulas, toddler milks, growing up formulas, maternal formulas, foods for active lifestyles, medical foods or supplements. Beverages may include dairy beverages, sports beverages, smoothies, protein fortified fruit or vegetable juices, drinking yoghurts, acid protein fortified beverages, liquid coffee, liquid tea, or a liquid beverage whiteners.

In various embodiments the food product may comprise nutrients that include vitamins and minerals. The recommended daily requirements of vitamins and minerals can be specified for various population subgroups. See for instance, Dietary Reference Intakes: RDA and Al for vitamins and elements, United States National Academy of Sciences, Institute of Medicine, Food and Nutrition Board (2010) tables recommended intakes for infants 0-6, 6-12 months, children 1-3, and 4-8 years, adults males (6 age classes), females (6 age classes), pregnant (3 age classes) and lactating (3 age classes). Concentrations of essential nutrients in the liquid nutritional composition can be tailored in the exemplary serve size for a particular subgroup or medical condition or application so that the nutrition and ease of delivery requirements can be met simultaneously.

In various embodiments the food product is in bar or solid moulded form. “Solid moulded form” means that the food product has been moulded into a shape that holds its form.

In various embodiments, the pH of the food product may be adjusted using food-safe acidic or basic additives. In various embodiments, the pH of the food product may be adjusted to about pH 3.5 to about 8, pH 4 to about pH 8, for example to about pH 4 to about pH 7, or about pH 4 to about pH 6.8, or about pH 5 to about pH 7, or about pH 5 to about pH 6.8. In various embodiments, the pH of the food product may be adjusted to about pH 6.8.

pH may be measured by equilibrating samples to 25° C. and measuring using a pH probe (EC620132, Thermo Scientific) after calibrating using standards at pH 4, 7, and 10 (Pronalys, LabServ). Other methods of measuring pH will be apparent to a skilled worker.

In various embodiments the food product may be administered to a subject to maintain or increase muscle protein synthesis, maintain or increase muscle mass, prevent or increase loss of muscle mass, maintain or increase growth, prevent or decrease muscle catabolism, prevent or treat cachexia, prevent or treat sarcopenia, increase rate of glycogen resynthesis, modulate blood sugar levels, increase insulin response to raised blood glucose concentration, increase satiety, increase satiation, increase food intake, increase calorie intake, improve glucose metabolism, increase rate of recovery following surgery, increase rate of recovery following injury, increase rate of recovery following exercise, increase sports performance, and/or provide nutrition.

In various embodiments, the food product may comprise at least about 0.1% fat by weight, such as about 0.1%, or about 0.5%, or about 1%, or about 3%, or about 5%, or about 10% fat by weight. In various embodiments, the food product may comprise from about 0.1% to 40% fat by weight, and useful ranges may be selected from between any of these values (for example, from about 0.1% to about 40%, or about 0.5% to about 40%, or about 1% to about 40%, or about 3% to about 40%, or about 5% to about 40%, or about 10% to about 40%, or about 15% to about 40%, or about 20% to about 40%, or about 0.1% to about 35%, or about 0.5% to about 35%, or about 1% to about 35%, or about 3% to about 35%, or about 5% to about 35%, or about 10% to about 35%, or about 15% to about 35%, or about 20% to about 35%, or about 0.1% to about 30%, or about 0.5% to about 30%, or about 1% to about 30%, or about 3% to about 30%, or about 5% to about 30%, or about 10% to about 30%, or about 15% to about 30%, or about 20% to about 30%, or about 0.1% to about 20%, or about 0.5% to about 20%, or about 1% to about 20%, or about 3% to about 20%, or about 5% to about 20%, or about 10% to about 20%, or about 15% to about 20%).

In various embodiments, the food product may comprise at least about 0.1% carbohydrate by weight, such as about 0.1%, or about 0.5%, or about 1%, or about 3%, or about 5%, or about 10% fat by weight. In various embodiments, the food product may comprise from about 0.1% to 40% carbohydrate by weight, and useful ranges may be selected from between any of these values (for example, from about 0.1% to about 40%, or about 0.5% to about 40%, or about 1% to about 40%, or about 3% to about 40%, or about 5% to about 40%, or about 10% to about 40%, or about 15% to about 40%, or about 20% to about 40%, or about 0.1% to about 35%, or about 0.5% to about 35%, or about 1% to about 35%, or about 3% to about 35%, or about 5% to about 35%, or about 10% to about 35%, or about 15% to about 35%, or about 20% to about 35%, or about 0.1% to about 30%, or about 0.5% to about 30%, or about 1% to about 30%, or about 3% to about 30%, or about 5% to about 30%, or about 10% to about 30%, or about 15% to about 30%, or about 20% to about 30%, or about 0.1% to about 20%, or about 0.5% to about 20%, or about 1% to about 20%, or about 3% to about 20%, or about 5% to about 20%, or about 10% to about 20%, or about 15% to about 20%).

In various embodiments, the food product is a nutritional formulation (for example a medical or sports beverage) and may comprise at least about 10 kcal per 100 mL of the food product. In various embodiments, the food product may comprise from about 10 to about 400 kcal per 100 mL of the food product, and useful ranges may be selected from between any of these values (for example, from about 10 to about 400, 10 to about 350, or about 10 to about 300, or about 10 to about 300, or about 10 to about 250, or about 10 to about 200, or about 10 to about 150, or about 10 to about 100, or about 50 to about 400, or about 50 to about 350, or about 50 to about 300, or about 50 to about 300, or about 50 to about 250, or about 50 to about 200, or about 50 to about 150, or about 50 to about 100, or about 100 to about 400, or about 100 to about 350, or about 100 to about 300, or about 100 to about 300, or about 100 to about 250, or about 100 to about 200, or about 100 to about 150, or about 150 to about 400, or about 150 to about 350, or about 150 to about 300, or about 150 to about 300, or about 150 to about 250, or about 200 to about 400, or about 200 to about 350, or about 200 to about 300, or about 200 to about 350).

In one embodiment the food product is in bar or solid moulded form. In various embodiments the bar further comprises one or more additional ingredients selected from one or more sweeteners, one or more additional protein sources, one or more binders (such as glucose syrup and/or high fructose corn syrup), one or more stability enhancers (such as glycerine and/or polyols), one or more plasticisers (such as glycerine and/or polyols), and/or one or more lipids.

In various embodiments the food product may be a shelf-stable protein beverage comprising 3.5% by weight of the plurality of recombinant proteins exhibits no visible sedimentation after 4 weeks at ambient temperature. “No visible sedimentation” means that no sedimentation is observed when the beverage is viewed unaided by the human eye in natural light.

In various embodiments, the food product has favourable coagulation properties when a composition described herein is added to one or more additional ingredients to produce the food product. In a preferred embodiment, when the composition is added to one or more additional ingredients, the resulting mixture does not rapidly coagulate.

In various embodiments, when the composition is added to a skim milk composition comprising 10% by weight total solids to a concentration of the one or more recombinant proteins of about 1% by weight and the skim milk composition is held at ambient temperature for 15 minutes then heated at 140° C., the composition has not coagulated after about 14, 15, 16, 17, 18, 19, 20, 25 or 30 minutes. The time to coagulation can be tested according to various methods known in the art, including those described herein. Time to coagulation can be measured by adding recombinant protein composition to skim milk, incubating for a certain period of time and then heating to a selected temperature.

In various embodiments, the food product has favourable gelation properties when a composition described herein is added to one or more additional ingredients to produce the food product. In a preferred embodiment, when the composition is added to one or more additional ingredients, the resulting mixture does not rapidly gel.

In various embodiments when the composition is added to a skim milk composition comprising 10% by weight total solids to a concentration of the one or more recombinant proteins of about 1% by weight, and the skim milk composition is heated at 40° C. for 12 hours, the skim milk composition does not reach gelation point. Gelation can be measured by various methods known in the art, including those as described herein. Briefly, a protein composition is added to skim milk and the sample is placed on a rheometer. Temperature is increased and the rheological properties of the sample are continuously monitored over a period of time. Gelation point can be detected by a change in the G′ from baseline.

The above methods should be considered in no way limiting and suitable variations or alternatives will be apparent to those skilled in the art.

### EXAMPLES

### Example 1

This example describes the production of Pichia pastoris strains expressing a recombinant β-lactoglobulin protein, and purification of the protein.

### 1. Preparation of β-Lactoglobulin a and β-Lactoglobulin B DNA Constructs

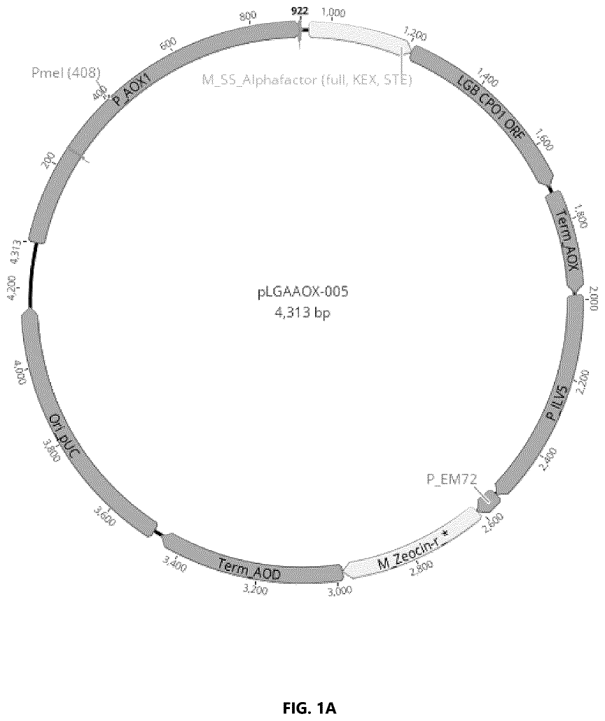

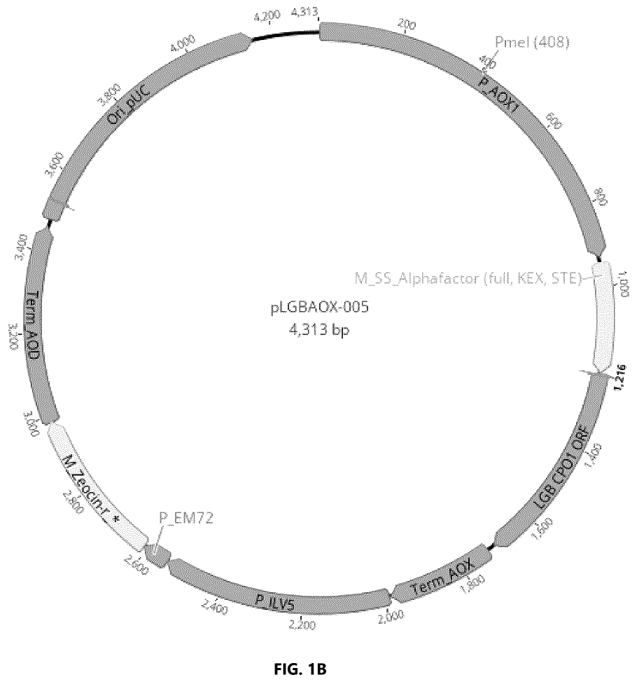

The proteins were expressed and produced using Pichia pastoris (currently renamed as Komagataella phaffii) host cells. Before transformation to the host cell, DNA constructs were designed and prepared as follows. The genes coding for native, mature bovine β-lactoglobulin A and β-lactoglobulin B were provided to order by synthetic DNA provider ATUM (CA, USA). Codon usage was optimized for expression in Pichia pastoris. The β-lactoglobulin A and β-lactoglobulin B encoding sequences were fused behind the α-mating factor from S. cerevisiae followed by a Kex2 processing site (KREAEAM) composed of lysine, arginine (KR), a glutamine-alanine repeat (EAEA) and a methionine at the start of the β-lactoglobulin A and B amino acid sequence. This led to the synthesis of the two DNA sequences described as the gene coding for beta-lactoglobulin A (SEQ ID No: 36) and the gene for beta-lactoglobulin B (SEQ ID No: 37). The genes were delivered cloned in the standard ATUM vector pD912 and by that placed under control of the methanol inducible AOX1 promoter. The delivered plasmid containing the beta-lactoglobulin A was named pLGAAOX-005 (SEQ ID No: 38) and the plasmid containing the beta-lactoglobulin B named pLGBAOX-005 (SEQ ID No: 39). Both plasmids are shown in FIG. 1.

2. Transformation of pLGAAOX-005 and pLGBAOX-005 to Pichia pastoris

The vectors pLGAAOX-005 and pLGBAOX-005 were digested with Pmel and Pichia pastoris strain NRRL-Y11430 (a wild type strain received from the ARS Culture Collection) was transformed with the digested DNA. Transformation procedure was performed according to condensed electroporation protocol using freshly prepared solutions (Lin-Cereghino, Biotechniques et al., (2005) 38, (1):44-48). Transformants were plated on YPDS agar plates with 1000 μg/mL Zeocin (YPDS: 1% yeast extract, 2% peptone, 2% glucose, 1M sorbitol, 2% agar) and incubated at 30° C. for 72 h. Single colonies were picked from the plates and transferred to 96-well MTP agar plates containing YEPhD agar with 500 μg/ml. (YEPhD agar 10 g/l Yeast extract, 20 g/l Phytone peptone, 20 g/l glucose, 15 g/l Oxoid agar, Zeocin added after autoclaving). MTP agar plates were incubated for 72 hours at 30° C. From the MTP agar plate transformants were inoculated into 200 μl YephD medium in HDWP using a pin and incubated overnight in an INFORS MTP incubator at 30° C., 750 rpm and 80% humidity. This was used as pre-culture for the protein production experiment.

3. Production of Recombinant β-Lactoglobulin A and B in P. pastoris

The pre-culture was used to inoculate the production medium. From the 200 μl pre-culture, 20 μl was inoculated into 2 ml BMM medium (0.2 M Potassium Phosphate buffer pH 6.8, 13.4 g/l Yeast Nitrogen Base, 0.4 mg/l Biotin) in a 24-deepwell plate covered with a breathable seal. Incubation of the plate was done in an INFORS MTP incubator at 28° C., 550 rpm and 80% humidity for 3 days. At the start and after 24 hours and 48 hours 1% of methanol was added for growth of the methanol utilizing Pichia pastoris strains and induction of the AOX1 promoter expressing the beta-lactoglobulin A and B genes. After 72 hours the 24-deepwell plates were centrifuged and 200 μl supernatant of the sample for each strain was transferred to a microtiter plate. The supernatant was analysed by LC-MS.

Food grade protein compositions were produced using a 10 litre scale fermenter, followed by downstream processing including removal of biomass by centrifugation, ultrafiltration for concentration and dialysis, anionic exchange for purification, ultrafiltration for concentration and dialysis for removal of salts and drying by freeze drying.

### 4. LC-MS/MS Analysis for Identification of PEP4

The following analysis was performed to demonstrate expression of the PEP4 protease.

For sample preparation of the supernatant samples, 10 μl BSA (10 g/l) was added to all samples. For protein precipitation, 210 μL of 20% TCA was added to the samples, and the solution was put at 4° C. for 1 hour. After precipitation of the proteins, the samples were centrifuged for 10 min at 2250 rcf at 4° C. The supernatant was removed and the pellet was washed once with 200 μl—20° C. acetone. The washed pellet was centrifuged for 10 min at 20817 rcf at 4° C. The supernatant was removed by drying with the N2 air for 10 min.

After drying the pellets, protein digestion was performed by re-dissolving the pellets in 50 μl 50 mM NaOH. 250 μL 100 mM NH4HCO3 was added. For reducing, 10 μl 250 mM DTT was added and incubated for 30 min at 55° C. in a thermomixer at 1000 rpm. For alkylation, 10 μl of 275 mM IAA was added and incubated for 30 min at room temperature in the thermomixer at 1000 rpm in the dark. Remaining IAA was quenched by placing all samples in the light for 10 min. 15 μl 0.25 μg/μl Trypsin was added and incubated over night at 37° C. in a thermomixer at 1000 rpm. The remaining digest was stored at 4° C.

The digested supernatant samples were analyzed on the Q Exactive plus (2) (Thermo Fisher), equipped with an Ultimate 3000 (Thermo Fisher). The chromatographic system consists of a UPLC CSH C18, 130 Å, 1.7 μm, 2.1 mm×50 mm+ACQUITY UPLC Col. In-Line Filter 0.2 μm, 2.1 mm, which is kept at 50° C. and gradient elution using A: 0.1% Formic Acid in water, and B: Formic Acid 0.1% in Acetonitrile, and a flow-rate of 400 ul/min. The gradient started at 5% B, then linear increasing to 35% B in 10 minutes, directly increasing to 80% B and kept here for 2 minutes, then directly decreasing to 5% B and kept here for 2 minutes, for re-equilibrating the LC-MS system.

The data was collected in Data Independent Acquisition (DIA) LC-MS/MS. Raw data was processed using Spectronaut 1.4 matching the Uniprot extraction from Komagataella pastoris (=P. pastoris), for identification of PEP4.

### Example 2

This example describes the production of Pichia pastoris strains having a pep4 knockout (Δpep4) expressing a recombinant β-lactoglobulin protein, and purification of the protein to produce compositions of the invention.

1. Plasmid pCASPP-05 Containing CAS9 and Guide RNA Targeting Pep4

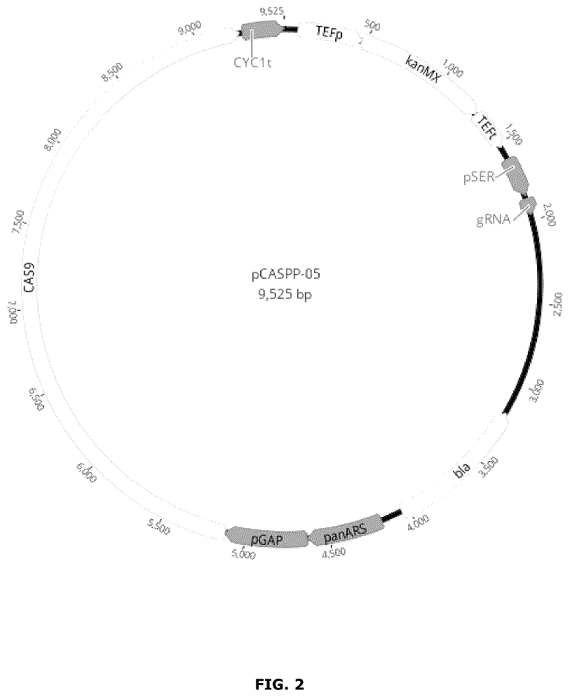

The functional knockout of pep4 was accomplished using CRISPR-CAS9 to support integration of a fragment encoding bovine β-lactoglobulin B and deletion of the pep4 gene sequence. An “all in one” plasmid named pCASPP-05 (shown in FIG. 2) was used, which expressed both CAS9 and the guide RNA (gRNA) targeting the pep4 sequence. The construction of pCASPP-05 (SEQ ID 40) was done using standard Gibson cloning methods. The plasmid contains the CAS9 gene expressed with the pGAP promoter and CYC1t terminator (SEQ ID 41) and the pep4 gRNA expressed with the pSER promoter (SEQ ID 42). The plasmid contains the kanMX selectable marker and an autonomously replicating sequence to select and maintain the plasmid in the cell after transformation to Pichia.

### 2. Preparing the β-Lactoglobulin B DNA PEP4 Integration Fragments

The gene coding for native, mature bovine β-lactoglobulin B was ordered at synthetic DNA provider ATUM (CA, USA). The β-lactoglobulin B encoding sequences were fused behind the α-mating factor from S. cerevisiae followed by a Kex2 processing site composed of lysine, arginine (KR) at the start of the β-lactoglobulin B amino acid sequence. The β-lactoglobulin B open reading frame was placed under control of the methanol inducible AOX1 promoter and the AOX1 terminator was placed at the end of the ORF forming the β-lactoglobulin B expression cassette (SEQ ID 43). The integration fragment transformed to Pichia was obtained by PCR amplification. The β-lactoglobulin B expression cassette (SEQ ID 43) was used as DNA template in the PCR. Forward primer DBC-26963 (SEQ ID 44) and reverse primer DBC-26964 (SEQ ID 45) were used to amplify the β-lactoglobulin B expression cassette attaching 60 bp flanking regions to facilitate homologous recombination in the genome and deleting the PEP4 DNA sequence. The pep4 deletion fragment is listed as SEQ ID 46. The PCR used Q5® High-Fidelity DNA Polymerase from NEW ENGLAND Biolabs and the protocol used is described in Tables 2 and 3. After PCR amplification, DNA was purified using the commercial PCR purification kit “DNA Clean & Concentrator Kits” from Zymo research.

TABLE 2
PCR protocol to amplify integration fragment

| | Volume (μl) | Final concentration | |
|---|---|---|---|
| 5X Q5 Reaction Buffer | 10 | 1X | |
| 10 mM dNTPs | 1 | 200 | μM |
| 10 μM Forward Primer | 2.5 | 0.5 | μM |
| 10 μM Reverse Primer | 2.5 | 0.5 | μM |
| Template DNA | 1 | 1 | ng |
| Q5 High-Fidelity DNA Polymerase | 0.5 | 0.02 | U/μl |
| Nuclease-Free Water | 32.5 | | |

TABLE 3
PCR protocol used to amplify integration fragment

| Temp (° C.) | Duration |
|---|---|
| 98 | 30 s |
| 98 | 10 s |
| 55 | 30 s |
| 72 | 2 min |
| Repeat previous 3 steps 5 times | |
| 98 | 10 s |
| 65 | 30 s |
| 72 | 2 min |
| Repeat previous 3 steps 35 times | |
| 72 C. | 2 min |

### 3. Transformation of the β-Lactoglobulin B DNA Pep4 Integration Fragments

The vector pCASPP-05 and the purified PCR fragment containing the β-lactoglobulin B expression cassette flanked with 50 bp integration flanks were transformed to the Pichia pastoris strain NRRL-Y11430 (a wild type strain received from the ARS Culture Collection). The amount of DNA used in the transformation for both plasmid and the integration fragment was 1 μg. Transformation procedure was performed according to condensed electroporation protocol using freshly prepared solutions (Lin-Cereghino et al. Biotechniques (2005) 38, (1):44-48). Transformants were plated on G418 Selective YEPDS agar (YPDS: 1% yeast extract, 2% peptone, 2% glucose, 1M sorbitol, 2% agar) with G418 added to a final concentration of 750 μg/ml. Plates were incubated at 30° C. for 72 h. Single colonies were picked from the plates and transferred to 96-well MTP agar plates containing YEPHD agar YEPhD-agar (BBL Phytone peptone 20.0 g/l, Yeast Extract 10.0 g/l, Sodium Chloride 5.0 g/l, Agar 15.0 g/l and 2% glucose) and 500 μg/ml G418. MTP agar plates were incubated for 72 hours at 30° C. From the MTP agar plate transformants were inoculated into 200 μl YEPHD medium (BBL Phytone peptone 20.0 g/l, Yeast Extract 10.0 g/l, Sodium Chloride 5.0 g/l and 2% glucose) in half deepwell plates (HDWP) using a pin and incubated overnight in an INFORS MTP incubator at 30° C., 750 rpm and 80% humidity. This was used as pre-culture for the protein production experiment.

4. Production of Recombinant β-Lactoglobulin B in P. pastoris ΔPep4 Host Cells

Recombinant β-lactoglobulin was produced in P. pastoris Δpep4 according to the method described above in Example 1.

Food grade protein compositions were produced using a 10 litre scale fermenter, followed by downstream processing including removal of biomass by centrifugation, ultrafiltration for concentration and dialysis, anionic exchange for purification, ultrafiltration for concentration and dialysis for removal of salts and drying by freeze drying.

### Example 3

This example describes methods for detecting aspartyl protease activity in recombinant milk protein compositions via detection of κ-casein degradation.

### 1. Methods

Skim milk (20% TS) was mixed with a recombinant protein composition produced as described in Example 1 or 2 above or with standard, milk-derived β-lactoglobulin and water to provide a skim milk content of 10% TS and a β-lactoglobulin content of 0, 0.25, 0.5, 0.75 or 1% w/w.

The milk samples were held for one hour to equilibrate. Sub-samples of each mixture were heated at 80° C. for 30 min or 120° C. for 10 minutes. The unheated and heated samples were held at 5° C. for 6 hours and then analyzed by traditional sodium dodecyl sulphate polyacrylamide gel electrophoresis (SDS PAGE) or “lab-on-a-chip” microfluidic SDS electrophoresis. Detailed methods for “lab-on-a-chip” is provided in S. G. Anema 2009, International Milk Journal 19 (2009) 198-204”.

### 2. Results—SDS PAGE

For the unheated samples with bovine milk-derived b-lactoglobulin, the gel showed no change in the intensity of the k-casein band and no appearance of a peptide peak in the region expected for para-k-casein. In contrast, the unheated samples comprising recombinant b-lactoglobulin produced in Pichia cells with the pep4 gene according to Example 1, the band intensity of the k-casein band decreased and a peptide band consistent with that of para-k-casein appeared. This effect was more pronounced as the level of b-lactoglobulin increased from 0.25 to 1.0%.

When bovine milk-derived b-lactoglobulin was heated, the gel also showed no change in the intensity of the k-casein band and no appearance of a peptide peak in the region expected for para-k-casein. In contrast, in the heated samples with the recombinant b-lactoglobulin produced in cells with the pep4 gene according to Example 1, the band intensity of the k-casein band decreased and a peptide band consistent with that of para-k-casein appeared. The conversion was more pronounced as the level of b-lactoglobulin increased from 0.25 to 1.0%, but was less pronounced than the unheated samples at each level of added recombinant b-lactoglobulin. The loss of the k-casein band and the appearance of a para-k-casein band were therefore clear indicators of aspartyl protease activity in the fermentation derived b-lactoglobulin preparation.

Unheated and heated subsamples with 0.5% fermentation derived b-lactoglobulin and an unheated sample with 0.5% milk derived b-lactoglobulin were held for 24 hours at 5° C. and then analysed by traditional or chip-based sodium dodecyl sulphate polyacrylamide gel electrophoresis. For both the unheated and heated samples with bovine milk derived b-lactoglobulin, the k-casein peak was of normal intensity and there was no peptide peak in the region expected for para-k-casein. For the samples with b-lactoglobulin produced in cells with the pep4 gene, the k-casein peak in both the heated and unheated samples had markedly reduced in size and a peak in the position expected for para-k-casein was present. The loss of k-casein and the para-k-casein peak was more pronounced for the unheated sample than the heated sample. The reduction of the k-casein peak intensity and the appearance of a para-k-casein peak were therefore clear indicators of aspartyl protease activity in the fermentation derived b-lactoglobulin preparations.

### 3. Results—Lab on a Chip

Results obtained for the following proteins held for 24 hours at 5° C. is presented in Table 4.
B-lac 1A (produced in a host cell with the pep4 gene according to Example 1)
B-lac 1B (produced in a host cell with the pep4 gene according to Example 1)
B-lac 2A (produced in a Δpep4 host cell according to Example 2)
B-lac 2B (produced in a Δpep4 host cell according to Example 2).

B-lac 1A was also heated at 80° C. and 120° C. for 30 and 10 minutes, respectively, then held at 5° C. for 24 hours. After the 80° C. treatment, the peak area was 249.48 and 0 for κ-casein and para-κ-casein, respectively. After 24 hours, the peak area was 115.2 and 35.82 for κ-casein and para-κ-casein, respectively, indicating that κ-casein had degraded by 54%. After the 120° C. treatment, the peak area was 249.6 and 0 for κ-casein and para-κ-casein, respectively. After 24 hours, the peak area was 179.4 and 16.12 for κ-casein and para-κ-casein, respectively, indicating that κ-casein had degraded by 72%.

TABLE 4
Lab on a chip results

Peak area

| Sample | Protein | 0 h | 24 h |
|---|---|---|---|
| B-lac 1A | κ-casein | 255 | 91.5 (64% |
| | | | degraded) |
| | para-κ-casein | 0 | 146.85 |
| | formed | | |
| B-lac 1B | κ-casein | 253.98 | 138.72 (46% |
| | | | degraded) |
| | para-κ-casein | 0 | 56.1 |
| | formed | | |
| B-lac 2B | κ-casein | 260.9 | 262.1 |
| | para-κ-casein | 0 | 0 |
| | formed | | |
| B-lac 2A | κ-casein | 251.2 | 251.8 |
| | para-κ-casein | 0 | 0 |
| | formed | | |

### Example 4

This example investigates the heat stability of recombinant milk protein compositions of the invention.

### 1. Unheated Beverage Storage Stability and Heat Stability

Skim milk (10% w/w total solids) comprising 1% w/w of recombinant s-lactoglobulin prepared. Sodium azide (0.02% w/v) was added as a preservative.

The recombinant β-lactoglobulin was provided by the following compositions prepared according to Examples 1 and 2.
B-lac 1A (expressed in a wild type host cell according to Example 1)
B-lac 1B (expressed in a wild type host cell according to Example 1)
B-lac 2A (expressed in a Δpep4 host cell according to Example 2)
B-lac 2B (expressed in a Δpep4 host cell according to Example 2)

Samples of each milk (˜1 mL) were placed in sealed glass bottles (Wheaton brand bottles with an 8 mL total volume) and held for different times (0.15, 4.5 or 16 hours) at ambient temperature (˜22° C.). After holding for the required times, the samples were tested for heat stability by placing the vials horizontally in a rack and then in an oil bath preset to 140° C. The samples were rocked at ˜10 cycles per minute which moved the milk from one end of the vial to the other. The samples were monitored until they visibly coagulated. The time at which they coagulated was recorded as the heat coagulation time. The results are provided in Table 5.

TABLE 5
Sample description, enzyme status and heat coagulation
times (HCT in minutes (m) and seconds (s)).

| | | Concentration | HCT at | HCT at | HCT at |
|---|---|---|---|---|---|
| | Genetic | of sample in | time | time | time |
| Sample | variant | skim milk | 0.15 h | 4.5 h | 16 h |
| B-lac 1A | A | 1% w/w | 12 m 53 s | 00 m 51 s | 00 m 01 s |
| B-lac 1B | B | 1% w/w | 13 m 53 s | 12 m 27 s | 10 m 31 s |
| B-lac 2A | A | 1% w/w | 19 m 54 s | 19 m 51 s | 19 m 01 s |
| B-lac 2B | B | 1% w/w | 16 m 50 s | 16 m 44 s | 16 m 05 s |

### Example 5

This example investigates the storage stability and gelation of beverages comprising recombinant milk protein compositions of the invention.

### 1. Unheated Beverage Storage Stability and Rennet-Like Gelation

Skim milk (10% w/w total solids) with 0.5 or 1% w/w fermentation-derived β-lactoglobulin added (with active enzymes or without active enzymes) was prepared. Sodium azide (0.02% w/v) was added as a preservative. A sample of each milk (˜1.4 mL) was placed on a rheometer plate set at 20° C. A 4 cm cone was lowered into position. Light mineral oil was placed around the perimeter of the sample to prevent drying. In addition, the water trap on the cone was filled with water and the water trap cover plates were placed over the sample. The rheometer was set to increase the temperature to 40° C. within 1 minute and monitor the rheological properties for up to 12 hours at 40° C. The samples were oscillated at a frequency of 0.1 Hz and with a strain of 0.025%. The rheological properties were monitored every 20 seconds for the duration of the run. The gelation point was considered the time when the G′ increased from the base line. Table 6 shows the gelation times of the samples tested.

TABLE 6
Sample description, enzyme status and gelation time.

| | | Concentration of | Aspartyl | |
|---|---|---|---|---|
| | Genetic | sample in skim | protease | |
| Sample | variant | milk | present | Gelation time |
| B-lac 1A | A | 0.5% w/w | yes | 220 min |
| B-lac 1A | A | 1% w/w | yes | 110 min |
| B-lac 2A | A | 0.5% | no | did not gel |
| B-lac 2A | A | 1% | no | did not gel |
| B-lac 1B | B | 1% | yes | 380 minutes |
| B-lac 2B | B | 1% | no | did not gel |

### Example 6

This example investigates the undesired proteolysis of recombinant β-lactoglobulin produced in a non PEP4 deleted host, compared to a wild type β-lactoglobulin without any enzymatic side activity resulting from PEP4.

### 1. Acid Gelation Proteolysis Example

### Method

Reconstituted skim milk of 20% total solids was prepared by mixing low heat skim milk powder with water, stirring until dissolved and equilibrating in the cold for at least 12 hours. The b-lactoglobulin (B-Lac 1A from example 1) solutions were prepared by mixing the freeze-dried b-LG powder with water, stirring until dissolved and adjusting the pH to ˜-7.0. The b-LG concentration was determined using UV spectrometry at 280 nm and the extinction coefficient of 0.95 cm²/g. The skim milk, b-LG solution and water were combined at ratios to give a 10% total solids skim milk with about 1% added b-LG. A sub sample of each milk (6 mL) was transferred to a glass vial, sealed, and then held for 0, 24 or 48 hours at room temperature. After holding for the desired time, the samples were heated at 80° C. for 30 min, with continuous rocking in an oil bath preset to 80° C. After heating, the samples were cooled in water until they were at room temperature. A sub-sample of the heated milk (4.9 g) and glucono δ-lactone (0.1 g) were mixed together to give a milk sample with 2% glucono-δ-lactone. The sample was immediately placed on a rheometer (TA AR 2000) with a cone (4 cm, 4°) and plate arrangement. The cone was lowered into position and the small-strain rheological properties were measured at 30° C. using a frequency of 0.1 Hz and a strain of less than 0.25%. The stiffness (G′) was measured every minute for 3 hours. The stiffness (G′) after 3 hours was recorded as the final stiffness of the set acid gel. After 3 hours, the temperature of the sample on the rheometer was reduced to 5° C. at a rate of 1° C./min. The yield properties of the set gel at 5° C. were determined by shearing the sample at a constants shear rate of 0.005 s⁻¹ and monitoring the stress and the strain. The stress increased to a maximum and then decreased as the acid gel yielded. The yield stress was the maximum stress obtained, and the yield strain was the strain at which the yield stress occurred. A sub sample of each milk was tested for proteolysis using microfluidic chip SDS-PAGE.

### 2. Results

### Proteolysis

The milk sample with B-Lac 1A (Pep 4 present) b-LG showed evidence of proteolysis on storage with a decrease in the band associated with k-casein, and a new peptide peak appearing. The decrease in the k-casein peak was more marked, and the peptide peak was more pronounced as the storage time increased. Milk with wild b-LG showed no evidence of proteolysis on holding for up to 48 hours.

### Gelation Time

For the milk sample with B-Lac 1A added, the time for the milk samples to gel decreased as the holding time before heating increased (Table 7). This indicates that the samples with more proteolysis (longer holding time) gelled at a higher pH than those with less proteolysis. The milk with wild b-LG gelled at the same time regardless of the holding time indicating that the pH for gelation was unchanged.

TABLE 7
Effect of holding time of milk samples with added
b-lactoglobulin before heating on the gelation
time of the milk on subsequent acidification.

| Holding time before heating | Gelation time (min) | Gelation time (min) |
|---|---|---|
| (hr) | B-Lac 1A | Wild b-LG |
| 0 | 20.6 | 16.1 |
| 24 | 18.4 | 16.5 |
| 48 | 16.9 | 16.1 |

### Final Stiffness (G′ in Pa)

For the milk sample with added B-Lac 1A, the final stiffness of the acid gel decreased as the holding time of the milk before heating and acidification increased. This indicates that a weaker acid gel was formed when more proteolysis was present in the milk before the milk was heated and acidified to form the gel. The final stiffness for the acid gels from milk with the wild b-LG was relatively unchanged and showed no trend with holding time indicating that the acid gel structure was not affected by holding the milk prior to acidification.

TABLE 8
Effect of holding time on the gelation time and
final stiffness of acid gels with added b-LGn

| Holding time before heating | Final G′ (Pa) | Final G′ (Pa) |
|---|---|---|
| (hr) | B-Lac 1A | Wild b-LG |
| 0 | 470 | 414 |
| 24 | 423 | 444 |
| 48 | 364 | 426 |

### Yield Strain and Yield Stress of the Gels

The yield strain for the gel from milk with added B-Lac 1A was the same regardless of the holding time of the milk before heating and acidification. The yield stress of the acid gels from milk with B-Lac 1A decreased as the holding time of the milk increased before the milk was heated and acidified (Table 9). This indicates that a weaker acid gel that was easier to break was formed when more proteolysis was present in the milk before the milk was heated and acidified to form the gel. For the acid gels from the wild b-LG, the yield strains and yield stresses showed no trend with the holding time for the milk.

TABLE 9
Effect of holding time on the yield strains and
yield stresses for acid gels with added b-LG

| Holding time | Yield | Yield | Yield | Yield |
|---|---|---|---|---|
| before | strain (—) | stress (Pa) | strain (—) | stress (Pa) |
| heating (hr) | B-Lac 1A | B-Lac 1A | Wild b-LG A | Wild b-LG A |
| 0 | 0.492 | 352.7 | 0.701 | 564.4 |
| 24 | 0.486 | 335.2 | 0.717 | 476.1 |
| 48 | 0.507 | 262.1 | 0.724 | 577.5 |

### Various Embodiments of the Invention

1. A food product comprising a composition comprising one or more recombinant polypeptides, wherein the composition is substantially free of aspartyl protease-like activity.
2. The food product of embodiment 1, wherein the food product comprises casein.
3. The food product of embodiment 1 or 2 wherein the food product comprises κ-casein.
4. The food product of any one of embodiments 1 to 3, wherein the composition comprises one or more recombinant milk polypeptides.
5. The food product of any one of embodiments 1 to 4, wherein the food product further comprises a non-dairy source of protein.
6. The food product of any one of embodiments 1 to 5, wherein the food product is selected from the group comprising a fermented food, a yoghurt, a soup, a sauce, a bar, a gel, a nutritional formulation, a beverage, a beverage whitener, a cheese, a dairy tofu and a dessert.
7. The food product of embodiment 6, wherein the nutritional formulation is selected from the group comprising an infant formula, a follow-on formula, a toddler milk, a growing up formula, a maternal formula, a food for active lifestyles, a medical food and a supplement.
8. The food product of embodiment 6, wherein the beverage is selected from the group comprising a dairy beverage, a sports beverage, a smoothie, a protein fortified fruit or vegetable juice, a drinking yoghurt, an acid protein fortified beverage, liquid coffee, liquid tea, and a liquid beverage whitener.
9. A method for preparing a food product, the method comprising
   a) providing a composition comprising one or more recombinant polypeptides, wherein the composition is substantially free of aspartyl protease-like activity, and
   b) mixing the composition with one or more additional ingredients to produce the food product, preferably wherein at least one ingredient comprises casein, most preferably, κ-casein.
10. Use of a composition comprising one or more recombinant polypeptides, wherein the composition is substantially free of aspartyl protease-like activity to produce a food product, preferably wherein the food product comprises casein, most preferably κ-casein.
11. A composition comprising one or more recombinant milk polypeptides, wherein the composition is substantially free of aspartyl protease-like activity.
12. The composition of embodiment 11, wherein the one or more recombinant milk polypeptides have at least 70% sequence identity to a wild type, mature milk protein.
13. The composition of embodiment 11 or 12, wherein the one or more recombinant milk polypeptides comprise one or more β-lactoglobulin proteins, α-lactalbumin proteins, casein proteins, lactoferrin proteins, or any combination of any two or more thereof.
14. The composition of any one of embodiments 11 to 13, wherein the wild type, mature milk protein has a sequence selected from any one of SEQ ID Nos: 1-27.
15. The composition of any one of embodiments 11 to 14, wherein the wild type, mature milk protein is a β-lactoglobulin protein having a sequence selected from any one of SEQ ID Nos: 1-11.
16. The composition of any one of embodiments 11 to 14, wherein the wild type, mature milk protein is
   a) an α-lactalbumin protein having a sequence of SEQ ID No: 12;
   b) a casein protein having a sequence selected from any one of SEQ ID Nos: 13-25; or
   c) a lactoferrin protein having a sequence of SEQ ID No: 26 or 27.
17. The composition of any one of embodiments 11 to 16, wherein the one or more recombinant milk polypeptides have at least 75%, 80%, 85%, 90%, 95% or 99% sequence identity, or have 100% sequence identity to the wild type, mature milk protein.
18. The composition of embodiment 11, wherein the one or more recombinant milk polypeptides have at least 70%, 80%, 90%, 95% or 99% sequence identity, or 100% sequence identity, to a polypeptide having a sequence selected from any one of SEQ ID Nos: 28 to 33.
19. The composition of any one of embodiments 11 to 18, wherein, when the composition is added to a skim milk composition comprising 10% by weight total solids to a concentration of the one or more recombinant polypeptides of about 1% by weight and the skim milk composition is heated at 80° C. for 30 minutes then held at 5° C. for 6 hours,
   a) no degradation of κ-casein is observed, and/or
   b) no production of para-κ-casein is observed.
20. The composition of any one of embodiments 11 to 19, wherein, when the composition is added to a skim milk composition comprising 10% by weight total solids to a concentration of the one or more recombinant polypeptides of about 1% by weight and incubated at ambient temperature for 24 hours, less than about 45%, less than about 25%, less than about 10% or less than about 1% of κ-casein in the skim milk composition is degraded to form para-κ-casein.
21. The composition of any one of embodiments 11 to 20, wherein, when the composition is added to a skim milk composition comprising 10% by weight total solids to a concentration of the one or more recombinant polypeptides of about 1% by weight and the skim milk composition is held at ambient temperature for 15 minutes then heated at 140° C., the composition has not coagulated after about 14 minutes.
22. The composition of any one of embodiments 11 to 21, wherein, when the composition is added to a skim milk composition comprising 10% by weight total solids to a concentration of the one or more recombinant polypeptides of about 1% by weight, and the skim milk composition is heated at 40° C. for 12 hours, the skim milk composition does not reach gelation point.
23. The composition of any one of embodiments 11 to 22, wherein the composition is substantially free of aspartyl protease
   a) consisting of an amino acid sequence of SEQ ID no. 34 or 35, or
   b) having at least 75%, 80%, 85%, 90%, 95% or 99% sequence identity to SEQ ID Nos: 34 or 35.
24. The food product of any one of claims 1 to 8, wherein the food product comprises a composition of any one of embodiments 11 to 23.
25. The method of claim 9, wherein the composition comprises a composition of any one of embodiments 11 to 23.
26. A host cell for producing one or more recombinant milk polypeptides, wherein
   a) the species of the host cell is selected from Pichia pastoris, Kluyveromyces lactis, Saccharomyces cerevisiae and Aspergillus niger,
   b) the host cell lacks an operative pep4 gene, or has been modified to produce less PEP4 protein than a wild type control cell, and
   c) the host cell expresses one or more milk polypeptides.
27. The host cell of embodiment 26, wherein the host cell genome comprises one or more integrated sequences that encode one or more milk polypeptides.
28. The host cell of embodiment 26, wherein the host cell comprises a polynucleotide encoding one or more milk polypeptides.
29. The host cell of embodiment 28, wherein the polynucleotide encodes
   a) a signal sequence,
   b) a leader sequence,
   c) a milk polypeptide, and
   d) a processing site selected from KR, KREA, KREAEA and KREAEAM located between the leader sequence and the sequence encoding the milk polypeptide.
30. A method for producing one or more recombinant milk polypeptides comprising
   a) culturing a host cell of any one of embodiments 26 to 29 in a culture medium under conditions sufficient to allow for expression of the one or more recombinant milk polypeptides, and
   b) isolating the one or more recombinant milk polypeptides from the culture medium.
31. A composition comprising one or more recombinant milk polypeptides, wherein the composition is produced according to the method of embodiment 30.
32. A kit for replacing a pep4 gene in a host cell with a sequence encoding one or more milk polypeptides, the kit comprising
   a) a polynucleotide expression vector encoding
      i) a marker to select for transformed host cells,
      ii) a sequence that enables autonomous replication of the polynucleotide expression vector in the host cell,
      iii) a first promoter sequence,
      iv) a Cas9 protein,
      v) a terminator sequence,
      vi) a second promoter sequence, and
      vii) a guide RNA targeting the pep4 gene; and
   b) a polynucleotide encoding
      i) 5′ and 3′ integration sequences,
      ii) a promoter sequence,
      iii) a leader sequence,
      iv) a milk polypeptide;
      v) a processing site selected from KR, KREA, KREAEA and KREAEAM located between the leader sequence and the milk polypeptide; and
      vi) a terminator sequence.

1. A food product comprising a composition comprising one or more recombinant polypeptides, wherein the composition is substantially free of aspartyl protease-like activity.

2. The food product of claim 1, wherein the food product comprises casein, optionally κ-casein.

3. The food product of claim 1, wherein the composition comprises one or more recombinant milk polypeptides.

4. The food product of claim 1, wherein the recombinant polypeptide is a β-lactoglobulin protein, optionally is bovine β-lactoglobulin.

5. The food product of claim 1, wherein the food product further comprises a non-dairy source of protein.

6. The food product of claim 1, wherein the food product is selected from the group comprising a fermented food, a yoghurt, a soup, a sauce, a bar, a gel, a nutritional formulation, a beverage, a beverage whitener, a cheese, a dairy tofu and a dessert.

7. A method for preparing a food product, the method comprising
a) providing a composition comprising one or more recombinant polypeptides, wherein the composition is substantially free of aspartyl protease-like activity, and
b) mixing the composition with one or more additional ingredients to produce the food product, optionally wherein at least one ingredient comprises casein, optionally κ-casein.

8. A product comprising a composition comprising one or more recombinant polypeptides, wherein the composition is substantially free of aspartyl protease-like activity to produce a food product, optionally wherein the food product comprises casein, optionally κ-casein.

9. A composition comprising one or more recombinant milk polypeptides, wherein the composition is substantially free of aspartyl protease-like activity, preferably wherein the one or more recombinant milk polypeptides comprise one or more β-lactoglobulin proteins, optionally wherein the β-lactoglobulin protein has a sequence selected from any one of SEQ ID Nos: 1-11, optionally wherein the β-lactoglobulin proteins are bovine β-lactoglobulin proteins.

10. The food product of claim 1, wherein the food product comprises a composition according to claim 9.

11. A host cell for producing one or more recombinant milk polypeptides, wherein
a) the species of the host cell is selected from Pichia pastoris, Kluyveromyces lactis, Saccharomyces cerevisiae and Aspergillus niger,
b) the host cell lacks an operative pep4 gene, or has been modified to produce less PEP4 protein than a wild type control cell, and
c) the host cell expresses one or more milk polypeptides, optionally β-lactoglobulin.

12. The host cell of claim 11, wherein the polynucleotide encodes
a) a signal sequence,
b) a leader sequence,
c) a milk polypeptide, and
d) a processing site selected from KR, KREA, KREAEA and KREAEAM located between the leader sequence and the sequence encoding the milk polypeptide.

13. A method for producing one or more recombinant milk polypeptides comprising
a) culturing a host cell of claim 11 in a culture medium under conditions sufficient to allow for expression of the one or more recombinant milk polypeptides, and
b) isolating the one or more recombinant milk polypeptides from the culture medium.

14. A composition comprising one or more recombinant milk polypeptides, wherein the composition is produced according to the method of claim 13.

15. A kit for replacing a pep4 gene in a host cell with a sequence encoding one or more milk polypeptides, the kit comprising
a) a polynucleotide expression vector encoding
i) a marker to select for transformed host cells,
ii) a sequence that enables autonomous replication of the polynucleotide expression vector in the host cell,
iii) a first promoter sequence,
iv) a Cas9 protein,
v) a terminator sequence,
vi) a second promoter sequence, and
vii) a guide RNA targeting the pep4 gene; and
b) a polynucleotide encoding
i) 5′ and 3′ integration sequences,
ii) a promoter sequence,
iii) a leader sequence,
iv) a milk polypeptide;
v) a processing site selected from KR, KREA, KREAEA and KREAEAM located between the leader sequence and the milk polypeptide; and
vi) a terminator sequence.
